(19) United States
(12) Patent Application Publication
(10) Pub. No.: US 2015/0093418 A1
(43) Pub. Date: Apr. 2, 2015

(54) EYE SWAB

(71) Applicant: Joker AG, Kerzers (CH)

(72) Inventors: Meinrad FLURY, Kerzers (CH); Benjamin Tak Kwong LEE, Shatin (HK); Wing Shong Thierry CHAN, Lam Tin (HK); Chun Ho WONG, Shatin (HK); Yi Ming GAN, Tai Po (HK)

(21) Appl. No.: 14/484,189

(22) Filed: Sep. 11, 2014

(30) Foreign Application Priority Data
Sep. 30, 2013 (CH) 01664/13

(51) Int. Cl. A61L 31/04; A61L 31/06; A61L 31/14; A61L 31/16

(52) U.S. Cl. CPC A61L 31/042; A61L 31/16; A61L 31/06; A61L 31/14
USPC 424/400; 523/105; 106/217.7; 106/15.05

(57) ABSTRACT

In accordance with the invention, with an eye swab for cleaning eyes of vertebrate animals, for example dog's eyes, is provided. The eye swab consists of a gel-like putty, wherein the gel-like putty is held together from a reversibly bonded, three-dimensional network formed of at least one natural or synthetic polymer, at least one cross-linking agent and glycerol.

### TECHNICAL FIELD

The invention relates to an eye swab for cleaning eyes of vertebrate animals, for example dog eyes.

### PRIOR ART

The eyes of pets are extremely sensitive areas and are vulnerable to dust, loose hair or other foreign bodies, which may enter the eyes with the wind or in other ways. In particular, dog owners clean the eyes of their pets often with a cloth wetted with lukewarm water in order to remove such accumulations of foreign bodies from the corners of the eyes in the region of the lacrimal glands. The foreign bodies and bacteria introduced therewith may otherwise lead to inflammation, pain and other complaints, which additionally cause increased tear production. The increased tear production in turn often leads to undesirable and unaesthetic tear stains, in particular for example on a light fur in the eye regions of the dog.

For humans, sterile, moist and non-fibrous cleaning cloths are known to clean and care for eyes. These are usually packaged individually and in a sterile manner and can be saturated with plant extracts, which have a soothing and antiseptic effect on the sensitive skin of the eye part and reduce swelling. Cleaning gels provided on a paper cloth or on the cleaning cloths are also known. With the use of cloths, however, there is a risk of injury upon contact with the cornea or sclera of the eye.

In order to clean eyes, eye baths are also used. These baths rinse the eyes and have a refreshing effect on dry and irritated eyes. They are often used by allergy sufferers in order to rinse out dust or pollen form the eyes, but are unsuitable for pets due to the awkward application.

A gel-like cleaning agent or a gel-like putty made of natural polygalactomannans with a proportion from 75 to 95% by weight of water for lint-free removal of solid particles is known from WO 02/055642. A development of this gel-like putty for the cleaning of pet paws is described in WO 2013/056972. This pet paw cleaning agent additionally contains a disinfecting solvent from the group of saturated, monovalent alcohols and lanolin.

### DISCLOSURE OF THE INVENTION

One object of the invention is to specify an eye swab for cleaning eyes of vertebrate animals, for example dog eyes, with which accumulations of dirt and where applicable tear stains can be removed gently and easily from the eyes and where applicable from the surrounding fur. Damage to the cornea or sclera of the eye due to possible contact with the eye swab is to be avoided. In accordance with a further object, the eye swab is to be created in such a way that it can be provided easily with inflammation-inhibiting or soothing substances in order to improve the comfort and the well-being of the pet. The eye swab, when used with pets, should also cause the pet less stress. A further object lies in the fact that the eye swab is to be reusable a number of times. It is also to be possible to enrich the eye swab with antiseptic substances.

In the context of the invention, the term “eye” is to be understood in particular to mean the area of the eyelid, the eyelashes and the nictitating membrane accessible from the outside, wherein the cornea and the sclera also would not sustain any damage upon contact with the eye swab.

The term “gel-like putty” is to be understood to mean a highly viscous material that is easily deformable and with which impurities on the surface can be kneaded into the interior of the material by some kneading movements. The gel-like putty can stretch and retract, but can also tear and be joined together again.

In one embodiment, the eye swab for cleaning eyes of vertebrate animals, for example dog's eyes, consists of a gel-like putty. The gel-like putty is held together from a reversibly bonded, 3-dimensional network formed of at least one natural or synthetic polymer, at least one cross-linking agent and glycerol. Such an eye swab is very flexible and soft and can be used without causing damage to the eye. The gel-like putty can be provided easily with inflammation-inhibiting, soothing and/or antiseptic substances, which are incorporated or embedded for this purpose in the 3-dimensional network and can be released in a metered manner.

The reversibly bonded, 3-dimensional network of the gel-like material is held together via different chemical equilibriums between the components (polymer, cross-linking agent and glycerol), which allow the connections between the at least one cross-linking agent, the polymers and the glycerol to be broken dynamically and re-bonded. Here, the system always moves back into equilibrium. Due to this property, the gel-like putty can stretch and retract, but also tear and join together again. A shape-memory due to the resilience is limited only to short-term and rapid deformation.

Due to the 3-dimensional network of the gel-like putty, the eye swab can be re-used a number of times since the “used” surface of the material is folded inwardly and a new “unused” surface is formed.

In a further embodiment, the gel-like putty may additionally contain polymer microspheres, preferably with a mean particle size of 100 to 150 micrometers. The polymer microspheres may be expanded polymer microspheres with a low density from 30 to 130 kg/m³, preferably 65 to 100 kg/m³. These may have, for example, a core formed of acrylonitrile copolymer with an inert coating formed of calcium carbonate (for example Dualite®, E065-135D, Henkel Cooperation, USA).

Expanded microspheres are particularly advantageous, since they contribute to a particularly soft, ductile and slightly gel-like putty, which increase the comfort during application and significantly reduce the risk of injury of the cornea or sclera.

In order to stabilize the chemical equilibrium of the 3-dimensional network, the gel-like putty can contain salts suitable for the production of buffer solutions, in particular sodium phosphates. The pH in the gel-like putty is generally set in a range from 6.8 to 8.0, preferably approximately 7.0 to 7.6.

The at least one natural or synthetic polymer can be selected from the group consisting of galactomannan, in particular hydroxypropyl guar (HPG) or carboxymethyl guar (CMG), chitosan, polymerized siloxane and combinations thereof. By way of example, chitosan can be assisted by the suitability thereof as an agent for binding or absorbing fats, and removing and absorbing contaminations and eye flow from the eyes.

The at least one polymer forms the actual 3-dimensional structure held together via reversible cross-linking agents, for example boron (as borax or boric acid) or the amphiphilic nanoparticles described in PCT/EP2013/058101. Glycerol, which can also cross-link in part with the other components, can be used primarily for water retention and the adjustment of the tackiness, such that the surface of the eye swab has sufficient moisture and does not adhere to sensitive parts of the eye upon contact. In addition, the eye swab is prevented from drying out quickly.

The gel-like putty may also comprise propylene glycol, which also increases water retention and prevents the eye swab form drying out.

The surface moisture and the tackiness of the gel-like putty can be adjusted in such a way that the harmful particles are absorbed easily and efficiently and in doing so are removed from the eyes.

In order to ensure an application of maximum length, the eye swab can be stored in a tightly closable container until the moment of re-use. The eye swab generally has a volume from 10 to 100 milliliters, preferably 20 to 50 milliliters.

Active ingredients suitable for eye care can be embedded in the gel-like putty of the eye cloth.

The gel-like putty may further have a water retention that is selected in such a way that excessive drying-out is prevented in spite of the formation of surface moisture.

The gel-like putty may contain ingredients selected from the group consisting of preservation agents, disinfection agents, dyes, pigments, essential oils or oil mixtures, fragrances and combinations thereof.

In a further embodiment of the eye cloth, the gel-like putty comprises the following components as basic composition:

| | Quantity (% by weight—w/w) |
|---|---|
| Polymer (preferably galactomannan and | 4.5-7.5% |
| chitosan) | |
| Boron (as borax or boric acid) | 0.1-0.5% |
| Glycerol | 4.8-7.2% |
| Propylene glycol | 4.8-7.2% |
| Polymer microspheres | 2-4% |
| Further ingredients | 1-2% |
| Water | Remainder (up to total 100%) |

1. An eye swab for cleaning eyes of vertebrate animals consisting of a gel-like putty, wherein the gel-like putty comprises a reversibly bonded, 3-dimensional network formed of at least one natural or synthetic polymer, at least one cross-linking agent and glycerol.

2. The eye swab according to claim 1, wherein the gel-like putty comprises polymer microspheres.

3. The eye swab according to claim 2, wherein the polymer microspheres have a mean particle size from 100 to 150 micrometers.

4. The eye swab according to claim 2, wherein the polymer microspheres have a density from 30 to 130 kg/m³.

5. The eye swab according to claim 2, wherein the polymer microspheres have a density from 65 to 100 kg/m³.

6. The eye swab according to claim 1, wherein the at least one natural or synthetic polymer is selected from the group consisting of: galactomannan, chitosan, polymerized siloxane, and combinations thereof.

7. The eye swab according to claim 1, wherein the gel-like putty has a water retention that is selected in such a way that excessive drying-out is prevented in spite of the formation of surface moisture.

8. The eye swab according claim 1, further comprising at least one active ingredient suitable for eye care embedded in the gel-like putty.

9. The eye swab according to claim 1, wherein the gel-like putty comprises at least one ingredient selected from the group consisting of: preservation agents, disinfection agents, dyes, pigments, essential oils or oil mixtures, fragrances, and combinations thereof.

10. The eye swab according to claim 1, wherein the gel-like putty comprises at least one salt suitable for the production of buffer solutions.

11. The eye swab according to claim 10, wherein the salt is a sodium phosphate.

12. The eye swab according to claim 1, wherein the pH of the gel-like putty is in a range from 6.8 to 8.0.

13. The eye swab according to claim 1, wherein the pH of the gel-like putty is in a range from 7.0 to 7.6.

14. The eye swab according to claim 1, wherein the eye swab has a volume from 10 to 100 milliliters.

15. The eye swab according to claim 14, wherein the eye swab has a volume from 10 20 to 50 milliliters.

16. The eye swab according to claim 1, wherein the gel-like putty comprises the following components:

| | | |
|---|---|---|
| polymer | 4.5-7.5% | by weight |
| boron (as borax or boric acid) | 0.1-0.5% | by weight |
| glycerol | 4.8-7.2% | by weight |
| propylene glycol | 4.8-7.2% | by weight |
| polymer microspheres | 2-4% | by weight |
| further ingredients | 1-2% | by weight, |

and water up to total 100% by weight.

17. The eye swab according to claim 16, wherein the polymer comprises galactomannan and chitosan.
